(19) United States
(12) Patent Application Publication
(10) Pub. No.: US 2022/0046939 A1
(43) Pub. Date: Feb. 17, 2022

(54) PLANT BASED MILK COMPRISING PROTEIN HYDROLYSATE AND DIVALENT CATION COMPOSITIONS HAVING IMPROVED TASTE AND STABILITY

(71) Applicant: Steuben Foods, Inc., Elma, NY (US)

(72) Inventor: Donkeun Park, Henrico, VA (US)

(73) Assignee: Steuben Foods, Inc., ELMA, NY (US)

(21) Appl. No.: 17/403,639

(22) Filed: Aug. 16, 2021

Related U.S. Application Data
(60) Provisional application No. 63/065,928, filed on Aug. 14, 2020.

(51) Int. Cl. A23C 11/10; A23G 9/38; A23G 9/36; A23G 9/42

(52) U.S. Cl. CPC A23C 11/10; A23C 11/103; A23V 2002/00; A23G 9/363; A23G 9/42; A23G 9/38

(57) ABSTRACT

Plant based beverage products and processes are disclosed, particularly plant based milk and creamer compositions comprising divalent cationic salts and treated with endoprotease and ionic compounds, including divalent cationic salts. In some embodiments, the process discloses a limited degree of protein hydrolysis in combination with added divalent cations. The process results in plant based milks with improved sensory and functional quality when compared to existing products, particularly reduced feathering when used as a creamer. The process is preferably used with plant based beverages processed with minimal disruption of the native protein structure. The resulting products have stability and functionality similar to that of dairy beverage products.

### TECHNICAL FIELD

The present disclosure relates to a process for modification of plant proteins for use in foods and beverages.

### BACKGROUND

Plant based beverages including milk and creamer have been in growing in popularity. Consumer concerns related to health and environmental protection, among other concerns, have created a demand for replacement of dairy beverages with plant based beverages. As a new industry, plant based beverage production has, however, experienced some challenges in matching the quality of traditional dairy products such as milk and creamers.

Some of these challenges relate to the differences between plant protein and dairy protein. Although protein is generally not the major ingredient in a coffee creamer, it does impact final product functionality. Proteins contribute to the viscosity, emulsion stability and solution stability of a coffee creamer. In coffee creamers, solution stability is often evaluated by “feathering.” Feathering, defined as the coagulation of creamer protein in coffee, decreases the consumer appeal of the coffee.

Structurally, dairy proteins are generally smaller and more soluble when compared to plant proteins, and have a more acceptable flavor. Additionally, due in part to their smaller size and structure, dairy proteins are less likely to coagulate and cause feathering when used in combination with acidic beverages such as coffee. Coagulation of plant based proteins may be caused by caffeic, chlorogenic and/or tannic acids, or other compounds present in a food or beverage product. Further, exposure to heat may also cause coagulation in water-soluble proteins. Additionally, the cost of dairy products is generally higher than for plant based products, and the environmental impacts more severe when compared to plant based products used for the same purpose. With regard to protein structure and its effects on functional properties of plant based proteins including feathering, larger proteins, in general, have lower solubility than smaller proteins due to a lower decrease in entropy upon precipitation.

Dairy products contain casein, a protein having extraordinarily high heat stability, making milk and milk based products highly stable at high temperature and resistant to many other destabilizing environmental factors. The stability of casein has been attributed to its disordered conformation and to the chaperone effects of casein protein molecules. Additionally, casein contains a high amount of calcium. Calcium ions are thought to have a key role in casein functionality and stability since it is widely believed that casein in micelles are bound together by calcium ions and hydrophobic interactions. Further, solubility of a casein molecule, k-casein, over a very broad range of calcium concentrations, is also believed to play a major role in the stabilization of the casein micelle.

In order to make plant based beverage products function more like dairy based beverage products, enzymatic hydrolysis using proteases has been widely employed. Proteolysis can improve the functionality of plant based proteins by reducing average molecular mass, exposing hydrophobic regions and by liberating ionizable groups. Further, protein hydrolysis can alter structure, texture and health related properties of plant proteins and improve solubility, water and fat holding capacity, gelation, foaming, feathering and emulsifying properties.

With regard to feathering in non-dairy creamers, U.S. Pat. Pub. No. 20110236545 to Brown et al. disclosed that protein hydrolysis with proteases can inhibit feathering under certain circumstances. Plant protein hydrolysis, however, is not known to increase the functionality of beverage products to a level equivalent to dairy.

A well-known problem when using protein hydrolysis to improve functionality in plant based products is hydrolysis of proteins typically produces a bitter flavor and other undesirable off notes. Bitterness is a negative attribute associated with most food protein hydrolysates. The development of biotechnological solutions for hydrolysate debittering is ongoing. To date, no universal solution to hydrolysate bitterness and off notes has been developed, although a number of methods have been implemented to ameliorate the problem. Practical solutions to hydrolysate debittering are likely to involve variations in enzymatic processing conditions and use of enzymes with targeted hydrolytic specificity.

U.S. Pat. Pub. No. 20150257411 to Janse discloses mild protein hydrolysis to extract nutrients from agro-sources while reducing bitterness. Janse recognized that “[t]he use of proteolytic enzymes mostly results in a bitter tasting product due to a high degree of hydrolysis with limited applications in food.” (Janse, [0002]). Janse used the protease Neutrase®, which has broad, rather than targeted specificity, in conjunction with relatively short incubation times to achieve a limited degree of hydrolysis (DH) to reduce bitterness. Similarly, U.S. Pat. No. 5,716,801 to Nielsen discloses use of protease and ultrafiltration to generateorganoleptically acceptable plant protein hydrolysates from plant based proteins. Nielsen discloses the use of Neutrase® or Alcalase® protease, both of which have broad hydrolytic specificity.

Protein hydrolysis, however, does not always result in increased bitterness or decreased flavor quality of the resulting hydrolysate. Some proteases have been identified or produced specifically to limit bitterness or flavor problems caused by hydrolysis. These proteases may have medium hydrolysis rates, may produce larger peptide fragments, and may have target specificity for sites that do not expose bitterness-producing amino acids, such as hydrophobic amino acids. For example, Neutrase® has a slower hydrolysis rate and produces larger protein fragments than enzymes such as Alcalase®. Flavourzyme® is a mixture of endo and exoproteases, as well as other enzymes such as amylase, that does not generate much, if any, bitterness in its hydrolysates. Trypsin and chymotrypsin have target specificity for amino acid sequences that tend to result in less bitter hydrolysates than some other enzymes.

It has been reported that for certain combination of proteases and substrates protein hydrolysis can reduce bitterness of a protein, although this is not widely observed. For example, Korean Pat. No. 100450617 to Lee discloses that the combination of Neutrase® or Flavourzyme® with a soy protein based formulation reduces bitterness and substantially improves overall flavor of a soy based ice cream. Soy hydrolysates are generally known to be bitter, which has limited their use in food products, however Lee disclosed an approximate increase of 4 to 8 on a 15 point flavor scale. In pea protein isolate (PPI) Garcia Arteaga reported that, on a scale of 1-7, “[a]fter 15 min of hydrolysis, Bromelain (2.4), Protamex® (2.5), Trypsin (2.6), and Papain (2.7) hydrolysates showed lower bitter intensities” when compared to the untreated PPI (3.0).

In contrast to Lee, however, Seo found that protein hydrolysis of soy protein isolate (SPI) increased bitterness regardless of the type of enzyme used, although certain enzymes generated much less bitterness (Seo et al., 2008). “As DH increased, the bitterness increased for all proteases evaluated. Alcalase® showed the highest TD factor at the same DH, followed by Neutrase®. Flavourzyme® showed the lowest TD factor at the entire DH ranges. At the DH of 10%, TD factor of hydrolysate by Flavourzyme® was 0 whereas those by Protamex® and Alcalase® were 4 and 16, respectively.” (Seo et al., 2008).

With regard to the proteases trypsin and chymotrypsin, Maehashi demonstrated that soy protein isolate hydrolyzed with trypsin does not cause bitterness, although, in contrast hydrolysis of SPI with the same amount of chymotrypsin over the same time period causes strong bitterness.

While it is clear from these studies that protein hydrolysis generally results in more bitterness and a reduction in sensory quality, in certain cases the results are less predictable. The results may depend on the type of protease employed as well as the protein substrate source. Selection of enzyme, reaction conditions, and substrate are not the only methods to reduce bitterness in protein hydrolysates, although they are well known.

To reduce bitterness in protein hydrolysates, different components (such as adenosine monophosphate) may be added to mask the effect of bitter taste (Sharma 2019). To overcome soy protein hydrolysate bitterness “xylitol, sucrose, α-cyclodextrin, maltodextrin and combinations of these were tested systematically as bitter masking agents” in an aqueous model. (Bertelson, 2018). In addition to masking agents “[m]ethods for debittering of protein hydrolyzates include selective separation such as treatment with activated carbon, extraction with alcohol, isoelectric precipitation, chromatography on silica gel, hydrophobic interaction chromatography” (Bertelson 2018), as well as other methods.

In the interest of limiting additives to produce clean label plant based products, masking agents are generally disfavored. Further, many of the physical or chemical methods described above are expensive and may require the use of undesirable chemicals. Therefore, it is clear that a need exists for an effective, inexpensive and clean label process for reducing bitterness and sensory problems caused by protein hydrolysis.

One potential circumstance where sensory problems with protein hydrolysates may occur is when protease is used with a plant based creamer to prevent feathering. Plant based creamers, which are particularly susceptible to feathering, generally require buffers and stabilizers to prevent coagulation. Buffers conventionally used in dairy or plant based creamers often contain a combination of an acid plus its salt, or a base plus its salt, and are used to maintain a stable pH in chemical and biological solutions. It is also common to add buffers to foods and beverages to stabilize particular proteins from precipitating/coagulating out in pH close to their isoelectric point and in hot beverages. Buffers exhibit little or no changes in pH with temperature and have maximum buffer capacity at a pH where the protein exhibits optimal stability.

Buffers and stabilizers frequently added to plant based creamers include gums, synthetic compounds, casein and casein derivatives (dairy protein derivatives), as well as whitening agents (Schmitt and Rade-Kukic, 2014; Schultz and Malone, 2020). Non-dairy powdered coffee creamers often contain stabilizers such as synthetic emulsifiers, buffer and stabilizing salts and may also contain whitening agents. Stabilizing additives may include buffer salts, chelators such as dipotassium phosphate, sodium citrate, disodium phosphate, potassium citrate, sodium citrate, calcium citrate, sodium hexametaphophate or a combination of the buffer salts to prevent feathering. Artificial and natural flavor combinations may also be added.

Addition of these artificially perceived food ingredients may be required to promote physical stability of the coffee creamer over the shelf life of the product and after pouring into coffee in order to achieve their desired whitening and flavor in the coffee. Without these conventional ingredients, plant based creamers are less effective, particularly in highly acidic coffee. Some creamer producers claim that their products are natural, however, they still contain these generally undesirable additives that are not considered to be clean label.

Phosphate buffers, some of the most common buffers used to prevent feathering, are of particular concern. Research has shown that phosphates can accumulate in the body and may cause organ calcification in people with renal failure as well as those with healthy kidney function (Ritz et al., 2012). While most buffers used in creamers are thought to be safe, researchers continue to uncover health problems associated with food additives that were previously considered harmless. The long term effects of many food additives are not fully understood, therefore health professionals as well as consumers have concerns about the use of such additives.

While some work has been done toward developing a plant based creamer that does not feather and is free from sensorial problems, other researchers have been seeking to improve the overall taste, or sensory properties, of plant based milk. “The main factors holding back the more widespread adoption of these products are their sensory attributes, stability, and functional performance (McClements, 2020). Consequently, producers are having to develop and test new formulations to meet consumer demands (Aydar et al., 2020; McClements, Newman, & McClements, 2019; Silva, Silva, & Ribeiro, 2020).” (McClements, 2021). “One of the main hurdles to widespread consumer adoption of plant-based milk alternatives is their taste and flavor profiles. Many consumers report the flavor of plant-based milks to have undesirable notes, such as “beany,” “bitter,” “astringent,” “grassy,” or “rancid” (Lawrence et al., 2016). Reducing these undesirable taste and flavor attributes are therefore important to increasing consumer acceptance.” (McClements, 2021).

Improvements in the taste of plant based milk, as well improvement in the ability of plant based milk to function like dairy milk, will be important in creating full commercial acceptance of plant based milk. Full consumer acceptance of plant based milk will allow society to realize the benefits of plant based food sources with regard to the environment and human health. Therefore, there is a need to improve the functionality of plant based milk while maintaining, or preferably improving, overall taste and providing the consumer with a clean label, healthy product.

### SUMMARY

In order to provide plant based beverage products with characteristics that have the desirable characteristics of dairy products, protease treatment of plant based milk, in combination with divalent cationic salts is disclosed. The plant based milk of the present disclosure may be produced from grains, nuts or seeds. Combinations of specific proteases and divalent cationic salts, when used in accordance with the process of the present disclosure, result in a plant based milk having unexpectedly good taste and functional properties.

In one embodiment, the enzyme is a serine endoprotease, such as trypsin or chymotrypsin, or trypsin like or chymotrypsin like serine endoproteases. Trypsin and chymotrypsin are known to cause milder hydrolysis than some other proteases. This property is desirable in the present disclosure in order to minimize negative effects on taste caused by hydrolysis. These proteases, when used according to the present disclosure, have a minimal degree of hydrolysis. This degree of hydrolysis, however, contributes to a surprisingly large improvement in feathering when combined with divalent cationic salts in accordance with the present disclosure. In addition, the minimal degree of hydrolysis according to the present disclosure causes a surprisingly large reduction in foaming, which has advantages during manufacturing and use of a creamer. In accordance with the present disclosure, the viscosity of these products is maintained at a low level that is acceptable for consumer use as a creamer. This viscosity may, in some embodiments, be approximately 500 cPs or lower when measured at a refrigerated temperature.

The present disclosure provides a plant based creamer that is capqable of preventing feathering at pH below 5.0, such as highly acidic coffee. Acidity and heat are two properties that are known to cause feathering in coffee creamers. Many creamers known in the art may prevent feathering in weaker coffee, however, the creamer of the present disclosure is capable of preventing feathering in very strong coffee where other plant based creamers would likely fail.

The divalent cationic salts of the present disclosure may be calcium cationic salts, including calcium carbonate, as well as combinations of calcium carbonate with magnesium and other compounds. In some embodiments, calcium cationic salts and magnesium cationic salts may be used in combination to meet nutritional requirements. Calcium may be preferable due to its molecular size and chemical properties, considering that magnesium and other similar divalent cation may be less ideal.

In one embodiment, the protease and divalent cationic salt are combined with the grains, nuts or seeds during the milking process. This milking process may involve wet milling of the grain, as described in U.S. Pat. No. 7,678,403 to Mitchell. Generally, in some embodiments of the present disclosure, it may be preferable to maintain the plant based protein in its native, non-denatured state protease hydrolysis. Gentle, wet milling the grain at low temperature to produce a plant based milk may be more effective in maintaining the native state of the protein than using flour or pressed grain, as is common in the industry, where high temperature and pressure can denature protein.

With regard to the process, the divalent cationic salt should be added such that it is present during activity of the protease. Generally, the divalent cationic salt should be added immediately prior to, or in conjunction with, the addition of the protease. The presence of the cationic salt during protein hydrolysis may control pH in a way that promotes desirable chemical reactions in order for the process to be effective.

### DETAILED DESCRIPTION

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. All references to percent are by weight.

The details of one or more embodiments of the invention are set forth in the accompanying drawings and the description below. Other features, objects, and advantages of the invention will be apparent from the description and drawings, and from the claims. Furthermore, although numerous details are set forth in order to provide a thorough understanding of the present invention, it will be apparent to one skilled in the art that these specific details are not required in order to practice the present invention. In other instances, details such as, well-known methods, types of data, protocols, procedures, components, networking equipment, processes, interfaces, electrical structures, circuits, etc. are not described in detail. All % values relating to formulations and concentration of components are by weight, where appropriate, unless specified otherwise.

Demand for plant based milk and other products traditionally made from dairy is growing. Many challenges remain for plant based food producers before full consumer acceptance of plant based dairy substitutes can be obtained. These challenges include producing a product that has functional and sensory properties that are equal to milk.

In one example of a generally inferior property of plant based beverages, plant based creamers are known to be more prone to feathering, or coagulation than dairy or dairy protein-based creamers. Factors that contribute to feathering of plant based creamers include protein size, which is generally larger in plant based products, and protein structure. The present disclosure alters protein structure in a manner that may contribute to its ability to improve feathering when used as a creamer.

As shown in Table 1 below, the combination of trypsin and calcium carbonate had a synergistic and unexpected effect on feathering reduction when added to strong coffee. In a preferred embodiment, the presence of trypsin and calcium carbonate were tested at effective concentrations for the process of the present disclosure. Trypsin was added to milk prepared according to Example 1 at a concentration of 0.04% w/w and calcium carbonate was added at a concentration of 0.25% w/w. These concentrations of trypsin and calcium carbonate comprise a preferred embodiment of the present disclosure.

TABLE 1
Effect of trypsin and calcium carbonate on oat milk and oat creamer

| | | | | Milk | Foam | | Creamer |
|---|---|---|---|---|---|---|---|
| | | Calcium | | Sensory | Quality^{ϕ¥} | Feathering | Viscosity |
| Trypsin | n | Carbonate | pH of Milk | Quality^{€} | (mL) | in Coffee^{¶} | (cPs) |
| None | 6 | None | 6.09 ± 0.10^{c} | 4.3 ± 0.3^{c} | 4.4 ± 0.5^{a} | 3.0 ± 0.6^{bc} | 278.3 ± 188.9^{a} |
| | | | | | (225.0 ± 17.7) | | |
| None | 6 | CaCO₃ | 7.31 ± 0.10^{a} | 5.5 ± 0.3^{b} | 3.0 ± 0.0^{b} | 3.4 ± 1.1^{b} | 136.2 ± 39.4^{a} |
| | | (1.0%) | | | (155.0 ± 11.2) | | |
| Trypsin | 6 | None | 6.04 ± 0.14^{c} | 4.7 ± 0.4^{c} | 4.8 ± 0.4^{a} | 2.7 ± 0.8^{c} | 225.3 ± 101.4^{a} |
| (0.1%) | | | | | (245.0 ± 27.4) | | |
| Trypsin | 6 | CaCO₃ | 7.17 ± 0.09^{b} | 6.8 ± 0.4^{a} | 2.0 ± 0.0^{c} | 4.8 ± 0.3^{a} | 171.4 ± 73.3^{a} |
| (0.1%) | | (1.0%) | | | (130.0 ± 11.2) | | |

^{a-c}Means with different letters in the same column are significantly different by Two-tailed T-test at p < 0.05.
^{€}Evaluated using 9 Point quality scale organoleptically: (1) Lowest Quality with lots of off notes and inferior quality aspects. (9) Highest quality without off notes, high intensity of intended flavor, right level of sweetness, mouthfeel, and good color.
^{ϕ}Evaluated using 5 Point quality scale from foam generated and observation of foam afterward. (1) Poor quality foam: Volume of milk/foam mix after foaming being 100-120 mL and the size of bubbles are big and collapse quickly. (2) Below average: Volume of milk/foam mix after foaming being 120-150 mL and the size of bubbles are big and collapse quickly. (3) Average: Volume of milk/foam mix after foaming being 125-175 mL with a mixture of big micro bubbles and collapse moderately. (4) Above Average: Volume of milk/foam mix after foaming being 150-200 mL with mostly micro foams and collapse slow. (5) Excellent: Volume of milk/foam mix after foaming being >200 mL with mostly micro foams and collapse slow.
^{¥}Results from n = 5 due to sample availability.

With regard to the sensory qualities of the product, as shown in the tables herein, the present disclosure was evaluated on a 9 point scale to measure organoleptic qualities of the product. On this scale, 1 is the lowest quality and represent a product with many off notes and generally inferior organoleptic properties. On this scale, 9 represents the highest quality product without off notes and having the appropriate flavor intensity, sweetness, mouthfeel and color.

With regard to foam quality, as shown in the tables herein, the present disclosure was evaluated using 5 Point quality scale from foam generated and observation of foam afterward. On a scale of 5, a score of 1 represents a poor quality foam. From a starting point of 100 mL of liquid, poor quality foam generally has a volume of milk and foam mix after foaming of between 100-120 mL where the size of bubbles are large and the bubbles collapse quickly. A score of 2 represents below average quality of milk, where the volume of milk and foam mix after foaming being 120-150 mL and the size of bubbles are large and the bubbles collapse quickly. A score of 3 represents average quality foam, where the volume of milk and foam mix after foaming is between 125-175 mL with a mixture of large micro bubbles and where the bubbles collapse moderately. A score of 4 represents above average quality milk, where the volume of milk and foam mix after foaming is between 150-200 mL including generally micro foam and where the bubbles collapse slowly. A score of 5 represents excellent quality foam, wherein the volume of milk and foam mix after foaming is greater than 200 mL and contains mostly micro foams and wherein the bubbles collapse slowly.

With regard to feathering, as shown in the tables herein, tables of the present disclosure used a 5 point feathering quality scale from formulated creamers that were added into hot, acidic (<5.0 pH) coffee and where feathering was observed after creamer was added to the coffee. A score of 1 represents a very unstable creamer, such that after addition of the creamer to coffee, the product feathered essentially instantly (<0.25 minutes). A score of 2 represents an unstable creamer, such that the creamer feathered in less than 3 minutes with large coagulations. A score of 3 represents an average quality creamer, with respect to feathering, such that the creamer feathered in 3-5 minutes after addition to the coffee. A score of 4 represents a semi-stable creamer, such that the creamer feathered between 5-10 minutes and the coagulation was very fine in size. A score of 5 represents a stable creamer, such that after addition to the coffee, the creamer did not feather for at least 10 minutes.

The unexpected, synergistic result on feathering is clearly shown in Table 1. Untreated oat milk has feathering score of 2.5 on a 5.0 scale. Oat milk treated with calcium carbonate only has a slightly higher feathering score of 3.0. Oat milk treated with trypsin only has a score of 2.5 on a 5.0 scale, showing no change from untreated milk. Based on this data, the expected feathering score for combined oat milk, trypsin and calcium carbonate would be 2.75. Surprisingly, however, the combined score of oat milk, trypsin and calcium carbonate prepared according to the process of the present disclosure was 4.5 out of 5.0. The actual improvement over the expected improvement was 1.75. This level of improvement is greater than expected and greater than additive, thus demonstrating an unexpected, synergistic effect on feathering reduction. While selection of protease and divalent cationic salt, as well as concentration of protease and divalent cationic salt, may vary within the scope of the present disclosure, in practice, the process of the present disclosure may be optimized within these parameters to achieve the unexpected, synergistic results using a wide variety of grains, nuts and seeds and in various products without departing from the scope and spirit of the present disclosure.

With regard to the process of the present disclosure, similar effects on feathering have been observed with soy, pea and other plant based milks or beverages. It is contemplated within the present disclosure that the process could be used with all grains, nuts and seeds that may be used to produce plant based milks.

U.S. Pat. Pub. No. 20110236545 to Brown disclosed a soy based creamer wherein use of trypsin like protease to hydrolyze soy protein isolate (SPI) caused a significant reduction in feathering when added to coffee. As shown in FIG. 5 of Brown, untreated SPI (SUPRO® 120) feathered when used in a creamer. Creamers using hydrolyzed SPI (SUPRO® 950 and SPP-A), however, had very little feathering. Brown did not disclose the addition of divalent cationic salts in combination with protease to achieve this effect.

Data from the present disclosure does not support a claim that the use of trypsin alone reduces feathering to any substantial degree. It is possible that Brown was using a relatively neutral pH coffee in its testing, in contrast to the present disclosure, which could explain a substantial feathering reduction. According to the results of the present disclosure and common knowledge in the art, it is much easier to reduce creamer feathering in weakly acidic coffee. Brown fails to disclose the pH of the coffee used for FIG. 5, and therefore does not its enable claims to feathering reduction, thereby making comparison of the feathering data to the present disclosure, where coffee is tested in highly acidic conditions below pH 5.0, impossible.

The Brown patent application was rejected based primarily on U.S. Pat. No. 5,024,849 to Rasilewicz, disclosing a whitener for liquid coffee that incorporated hydrolyzed soy protein in its formulation to improve taste; U.S. Pat. No. 4,100,024 to Adler-Nissen, disclosing an enzyme hydrolyzed soy protein for improved flavor as a food additive; and U.S. Pat. No. 6,465,209 to Blinkovsky et al., disclosing a method of producing a protein hydrolysate having a specified degree of hydrolysis and good flavor. Rasilewicz tested for feathering after 2 minutes, which is too short for practical observation of feathering in coffee. None of these references disclose a protein hydrolysate used in combination with a divalent cationic salt, as in the process of the present disclosure.

The effect on feathering, as well as other characteristics of plant based milk are dependent upon the conditions used in processing. The feathering reduction observed is dependent upon the type of enzymes and ionic compounds used in the process, the concentration of the components in the plant based milk. In some cases, concentration of enzyme and divalent cationic salts are herein listed as ranges, as disclosed in Table 2 below.

With regard to the effective ranges of the preferred embodiment for feathering reduction, wherein the reaction conditions were held constant to those described in Example 1, and when only protease concentration was varied, an effective range of trypsin concentration was preferably from approximately 0.01 to 0.30, or more preferably from approximately 0.04 to 0.30. These ranges can be correlated to a degree of hydrolysis (DH) by maintaining constant conditions, as described in Example 1, while measuring DH according to methods that are well known in the art, including the pH-stat, trinitrobenzenesulfonic acid (TNBS), o-phthaldialdehyde (OPA), trichloroacetic acid soluble nitrogen (SN-TCA), and formol titration methods. Therefore, for the purposes of the present disclosure, in one aspect the present disclosure can be claimed in a range of DH, ranging from a DH measured under the conditions of Example 1 from a concentration of trypsin of approximately 0.01 to 0.30% w/w, or preferably from approximately 0.04 to 0.30% w/w, or preferably from approximately 0.04 to 0.1% w/w. Table 2 shows that over a wide range of concentrations and ingredient type, many of which are suboptimal, the average effect on feathering when components are used under suboptimal conditions may be positive or negative.

Under optimal conditions, however, it is observed that feathering can be significantly reduced in comparison to creamer produced from untreated plant based milk. Table 2 also shows a general trend that with certain conditions, enzymes and ionic compounds, including monovalent, divalent and multivalent cationic salts, certain components of the formulation perform better than others with regard to feathering reduction. For example, over a wide range of concentrations, both suboptimal and optimal, Table 2 shows that the combination of trypsin and calcium carbonate performs better than virtually all combinations of ionic compounds and trypsin and other proteases.

Table 2 also shows that optimal concentrations and components can result in unexpected, synergistic improvements in feathering reduction when added to highly acidic coffee. In general, Table 2 shows that certain concentrations of a limited number of component combinations can result in a surprising improvement in feathering, as well as other characteristics of plant based milk and creamer. In order for a creamer to be acceptable to consumers, the viscosity must generally be below 500 cPs, as measured according to the method described in the present disclosure. An creamer without treatment according prepared according to the present disclosure may, in some embodiments, generally have a viscosity of approximately 1000 cPs. Interestingly, the addition of calcium carbonate alone significantly reduces viscosity, by about 75%, according to the process of the present disclosure. Unlike processes that combine calcium carbonate with plant based milk known in the art, which generally fortify plant based milk with calcium carbonate for nutritional purposes after the milk is prepared and fully hydrolyzed with amylase, the present disclosure adds calcium carbonate to the milk prior to any processes that may substantially denature proteins, such as dry milling or pressing, and without the milk having been fully hydrolyzed with amylase. According to one embodiment of the process of the present disclosure, amylase is only used to minimally digest starch, such that the starch can be high temperature processed, and calcium carbonate significantly reduces viscosity in plant based milk that has only been minimally hydrolyzed with amylase. Therefore, in plant based milk that have substantially native proteins and starch that has not been highly digested by amylase, calcium carbonate addition lowers viscosity in a manner that is of practical use in coffee creamers and plant based milk that benefit from viscosity reduction.

Prior studies have added calcium carbonate to plant based milks, however, none have disclosed any effect on viscosity from the addition. For example, U.S. Pat. 20140044855 to Sher discloses the addition of divalent cations to soy milk creamer for the purpose of whitening, however, no effect on viscosity was disclosed. In Sher, calcium carbonate was added to a pre-prepared creamer that included hydrocolloid and other components that could affect viscosity, as well as with soy protein from a flour that had been prepared by dry milling, or other methods that would have caused protein denaturation. Therefore, from Sher it can be seen that the addition of calcium carbonate to a plant based beverage product, even one that has hydrolyzed protein, does not necessarily cause viscosity reduction. Further, calcium carbonate is not known to be a viscosity reducing agent. Therefore, the demonstration of the present disclosure that calcium carbonate alone can reduce viscosity in oat milk and creamer is unexpected. The timing of addition of calcium carbonate is critical to observe this effect. Table 2 also includes data wherein alkaline protease is disclosed, and wherein the alkaline protease is chymotrypsin.

TABLE 2
Effect of ionic compounds and proteases on oat milk and oat creamer

| | | | Chemical | | | | Feathering | Creamer |
|---|---|---|---|---|---|---|---|---|
| Treatment | | Protease added | added | | Milk | Foam | in | Viscosity |
| Abbreviation | n | (Quantity) | (Quantity) | pH of Milk | Quality | Quality | Coffee | (cPs) |
| CaCbALKP | 1 | Alkaline | CaCO₃ | 7.08 ± 0.00 | 8.0 ± 0.0 | 4.0 ± 0.0 | 5.0 ± 0.0 | 142.7 ± 0.0 |
| | | Protease | (0.25%) | | | | | |
| | | (0.04%) | | | | | | |
| CaCbMgOTRY1 | 1 | Trypsin | CaCO₃, MgO | 7.33 ± 0.00 | 5.5 ± 0.0 | 4.0 ± 0.0 | 5.0 ± 0.0 | 429.3 ± 0.0 |
| | | (0.04%) | (0.25, 0.125%) | | | | | |
| CaCbTRY1AL | 1 | Trypsin, | CaCO₃, | 7.42 ± 0.00 | 8.0 ± 0.0 | 4.0 ± 0.0 | 5.0 ± 0.0 | 245.3 ± 0.0 |
| KP | | Alkaline | Ca(OH)₂ | | | | | |
| | | Protease | (0.25, 0.05%) | | | | | |
| | | (0.02, 0.02%) | | | | | | |
| CaHyTRY1 | 3 | Trypsin | Ca(OH)₂ | 8.96 ± 0.08 | 5.7 ± 0.6 | — | 4.3 ± 1.2 | 548.0 ± 597.5 |
| | | (0.04%) | (0.25%) | | | | | |
| CaCb | 3 | None | CaCO₃ | 6.99 ± 0.14 | 6.3 ± 1.5 | 4.0 ± 0.0 | 4.0 ± 1.0 | 288.0 ± 70.5 |
| | | (0.00%) | (0.25%) | | | | | |
| CaCbNEUT | 1 | Neutral | CaCO₃ | 7.09 ± 0.00 | 6.0 ± 0.0 | 4.0 ± 0.0 | 4.0 ± 0.0 | 162.7 ± 0.0 |
| | | Protease | (0.25%) | | | | | |
| | | (0.04%) | | | | | | |
| TRY1 | 5 | Trypsin | None | 6.21 ± 0.14 | 4.6 ± 0.8 | — | 3.8 ± 1.3 | 718.3 ± 566.1 |
| | | (0.04%) | (0.00%) | | | | | |
| NaClTRY1 | 4 | Trypsin | NaCl | 6.31 ± 0.13 | 5.0 ± 1.4 | — | 3.8 ± 1.0 | 619.3 ± 430.4 |
| | | (0.04%) | (0.13%) | | | | | |
| None | 4 | None | None | 6.24 ± 0.13 | 5.0 ± 0.8 | — | 3.8 ± 1.3 | 1028.3 ± 984.4 |
| | | (0.00%) | (0.00%) | | | | | |
| CaCbTRY1 | 6 | Trypsin | CaCO₃ | 7.15 ± 0.27 | 6.6 ± 0.8 | 2.8 ± 1.1 | 3.6 ± 1.1 | 311.0 ± 119.4 |
| | 2 | (0.01-0.3%) | (0.05-2.5%) | | | | | |
| MgCbTRY1 | 8 | Trypsin | MgCO3 | 8.24 ± 0.70 | 5.5 ± 1.1 | 1.8 ± 0.4 | 3.4 ± 1.4 | 332.5 ± 202.7 |
| | | (0.04%) | (0.1-2.0%) | | | | | |
| AlHyTRY1 | 6 | Trypsin | Al(OH)₃ | 6.62 ± 0.31 | 5.7 ± 1.0 | 3.3 ± 0.5 | 3.2 ± 1.0 | 371.0 ± 276.3 |
| | | (0.04%) | (0.1-2.0%) | | | | | |
| DCPTRY1 | 6 | Trypsin | CaHPO₄ | 6.30 ± 0.05 | 5.9 ± 0.8 | 2.3 ± 0.5 | 3.2 ± 0.4 | 340.0 ± 70.7 |
| | | (0.04%) | (0.1-2.0%) | | | | | |
| MPPTRY1 | 6 | Trypsin | KH₂PO₄ | 5.99 ± 0.13 | 5.0 ± 1.3 | 2.2 ± 1.5 | 3.2 ± 0.4 | 396.2 ± 143.8 |
| | | (0.04%) | (0.1-2.0%) | | | | | |
| TCPTRY1 | 6 | Trypsin | Ca₃(PO₄)₂ | 6.35 ± 0.06 | 5.8 ± 0.4 | 3.0 ± 0.8 | 3.2 ± 1.2 | 515.1 ± 291.6 |
| | | (0.04%) | (0.1-2.0%) | | | | | |
| CaCbPAPN | 1 | Papain | CaCO₃ | 7.22 ± 0.00 | 7.0 ± 0.0 | 4.0 ± 0.0 | 3.0 ± 0.0 | 348.0 ± 0.0 |
| | | (0.04%) | (0.25%) | | | | | |
| CaClTRY1 | 4 | Trypsin | CaCl₂ | 5.84 ± 0.12 | 4.5 ± 0.6 | — | 3.0 ± 1.4 | 561.0 ± 296.5 |
| | | (0.04%) | (0.25-0.28%) | | | | | |
| CaLtTRY1 | 2 | Trypsin | Ca Lactate | 5.85 ± 0.03 | 5.0 ± 1.4 | — | 3.0 ± 1.4 | 657.0 ± 360.2 |
| | | (0.04%) | (0.25%) | | | | | |
| MgHyTRY1 | 8 | Trypsin | Mg(OH)₂ | 8.73 ± 0.60 | 5.3 ± 1.6 | 2.0 ± 0.7 | 3.0 ± 1.7 | 482.7 ± 264.5 |
| | | (0.04%) | (0.1-2.0%) | | | | | |
| MgOALKP | 1 | Alkaline | MgO | 8.44 ± 0.00 | 6.0 ± 0.0 | 3.0 ± 0.0 | 3.0 ± 0.0 | 521.3 ± 0.0 |
| | | Protease | (0.25%) | | | | | |
| | | (0.04%) | | | | | | |
| MgONEUT | 1 | Neutral | MgO | 6.43 ± 0.00 | 5.0 ± 0.0 | 4.0 ± 0.0 | 3.0 ± 0.0 | 456.0 ± 0.0 |
| | | Protease | (0.25%) | | | | | |
| | | (0.04%) | | | | | | |
| MgOPAPN | 1 | Papain | MgO | 6.49 ± 0.00 | 6.0 ± 0.0 | 3.0 ± 0.0 | 3.0 ± 0.0 | 368.0 ± 0.0 |
| | | (0.04%) | (0.25%) | | | | | |
| ZnGlTRY1 | 2 | Trypsin | Zn Gluconate | 5.92 ± 0.07 | 5.0 ± 0.0 | 1.0 ± 0.0 | 3.0 ± 1.4 | 366.7 ± 92.4 |
| | | (0.04%) | (0.25%) | | | | | |
| CaClCaHyTRY1 | 4 | Trypsin | CaCl₂, | 7.30 ± 0.57 | 4.5 ± 1.3 | 1.3 ± 0.5 | 2.8 ± 1.5 | 196.0 ± 37.1 |
| | | (0.04%) | Ca(OH)₂ | | | | | |
| | | | (0.1-2.0%) | | | | | |
| MgOTRY1 | 7 | Trypsin | MgO | 6.36 ± 0.19 | 5.9 ± 0.6 | 3.6 ± 0.5 | 2.7 ± 0.8 | 462.5 ± 121.0 |
| | | (0.04%) | (0.1-2.0%) | | | | | |
| DMPTRY1 | 6 | Trypsin | MgHPO₄ | 6.41 ± 0.18 | 5.4 ± 0.5 | 1.3 ± 0.5 | 2.7 ± 1.2 | 522.0 ± 150.8 |
| | | (0.04%) | (0.1-2.0%) | | | | | |
| CaGlTRY1 | 2 | Trypsin | Ca Gluconate | 6.04 ± 0.13 | 6.0 ± 1.4 | — | 2.5 ± 2.1 | 618.0 ± 331.6 |
| | | (0.04%) | (0.25%) | | | | | |
| CaCl | 3 | None | CaCl₂ | 5.81 ± 0.14 | 4.8 ± 0.8 | 1.0 ± 0.0 | 2.0 ± 0.0 | 518.0 ± 423.8 |
| | | (0.00%) | (0.25-0.28%) | | | | | |
| CaClKOHTRY1 | 4 | Trypsin | CaCl₂ | 7.04 ± 0.04 | 5.4 ± 1.3 | 1.8 ± 1.5 | 2.0 ± 0.0 | 122.0 ± 30.2 |
| | | (0.04%) | (0.1-2.0%) | | | | | |
| MgClTRY1 | 2 | Trypsin | MgCl₂ | 5.97 ± 0.19 | 5.5 ± 0.7 | — | 2.0 ± 1.4 | 596.0 ± 311.1 |
| | | (0.04%) | (0.25%) | | | | | |
| TSPTRY1 | 6 | Trypsin | Na3PO₄ | 7.28 ± 0.85 | 5.8 ± 2.3 | 4.3 ± 1.0 | 1.8 ± 1.0 | 171.1 ± 140.0 |
| | | (0.04%) | (0.1-2.0%) | | | | | |
| DPPTRY1 | 6 | Trypsin | K2HPO₄ | 6.74 ± 0.36 | 5.7 ± 0.6 | 4.0 ± 1.2 | 1.8 ± 1.3 | 232.0 ± 74.8 |
| | | (0.04%) | (0.1-2.0%) | | | | | |
| MgCtTRY1 | 6 | Trypsin | Mg Citrate | 6.10 ± 0.13 | 4.8 ± 0.6 | 1.0 ± 0.0 | 1.7 ± 0.8 | 421.9 ± 310.7 |
| | | (0.04%) | (0.1-2.0%) | | | | | |
| CaOTRY1 | 5 | Trypsin | CaO | 9.69 ± 1.63 | 4.0 ± 2.5 | 1.8 ± 1.3 | 1.6 ± 0.9 | 5075.0 ± 4569.1 |
| | | (0.04%) | (0.1-2.0%) | | | | | |
| CaCtTRY1 | 6 | Trypsin | Ca Citrate | 6.01+ 0.09 | 5.6 ± 0.8 | 1.3 ± 0.5 | 1.5 ± 0.8 | 549.0 ± 567.1 |
| | | (0.04%) | (0.1-2.0%) | | | | | |
| KCbTRY1 | 6 | Trypsin | K₂CO₃ | 8.31 ± 1.17 | 4.8 ± 2.3 | 2.8 ± 2.1 | 1.3 ± 0.8 | 202.0 ± 91.4 |
| | | (0.04%) | (0.1-2.0%) | | | | | |
| MCPTRY1 | 5 | Trypsin | Ca(H₂PO₄)₂ | 5.65 ± 0.40 | 4.8 ± 2.3 | 1.2 ± 0.4 | 1.2 ± 0.4 | 227.9 ± 104.8 |
| | | (0.04%) | (0.1-2.0%) | | | | | |
| KOHTRY1 | 3 | Trypsin | KOH | 7.24 ± 0.23 | 6.3 ± 1.5 | 4.0 ± 0.0 | 1.0 ± 0.0 | 221.0 ± 105.2 |
| | | (0.04%) | (0.01-0.05%) | | | | | |
| NaCbTRY1 | 6 | Trypsin | Na₂CO₃ | 8.53 ± 1.08 | 4.0 ± 2.8 | 2.3 ± 2.6 | 1.0 ± 0.0 | 254.3 ± 70.9 |
| | | (0.04%) | (0.1-2.0%) | | | | | |

Table 3 provides additional data, similar to Table 2, wherein the effect of combinations of ionic compounds are disclosed.

TABLE 3
Average effect of different ionic compounds over a range of
concentrations on oat milk and oat creamer

| Ionic | | Quantity | | | Milk | | | Creamer |
|---|---|---|---|---|---|---|---|---|
| compounds | | of Ionic | Quantity of | pH of | Sensory | Foam | Feathering | Viscosity |
| added | n | Compounds | Proteases | Milk | Quality | Quality | in Coffee | (cPs) |
| CaCO₃, | 1 | (0.25, 0.05%) | (0.04%) | 7.42 ± 0.00 | 8.0 ± 0.0 | 4.0 ± 0.0 | 5.0 ± 0.0 | 245.3 ± 0.0 |
| Ca(OH)₂ | | | | | | | | |
| CaCO₃, MgO | 1 | (0.25, 0.125%) | (0.04%) | 7.33 ± 0.00 | 5.5 ± 0.0 | 4.0 ± 0.0 | 5.0 ± 0.0 | 429.3 ± 0.0 |
| CaCl₂, | 1 | (0.1%, 0.1%) | (0.04%) | 7.30 ± 0.00 | 6.0 ± 0.0 | 2.0 ± 0.0 | 5.0 ± 0.0 | 196.7 ± 0.0 |
| Ca(OH)₂ | | | | | | | | |
| Ca(OH)₂ | 3 | (0.25%) | (0.04%) | 8.96 ± 0.08 | 5.7 ± 0.6 | — | 4.3 ± 1.2 | 548.0 ± 597.5 |
| none | 9 | (0.00%) | (0.0-0.04%) | 6.22 ± 0.13 | 4.8 ± 0.8 | — | 3.8 ± 1.2 | 856.0 ± 741.9 |
| NaC1 | 4 | (0.13%) | (0.04%) | 6.31 ± 0.13 | 5.0 ± 1.4 | — | 3.8 ± 1.0 | 619.3 ± 430.4 |
| CaCO₃ | 68 | (0.05-2.5%) | (0.01-0.3%) | 7.15 ± 0.26 | 6.6 ± 0.9 | 2.9 ± 1.1 | 3.6 ± 1.1 | 305.5 ± 117.9 |
| MgCO₃ | 8 | (0.1-2.0%) | (0.04%) | 8.24 ± 0.70 | 5.5 ± 1.1 | 1.8 ± 0.4 | 3.4 ± 1.4 | 332.5 ± 202.7 |
| A1(OH)₃ | 6 | (0.1-2.0%) | (0.04%) | 6.62 ± 0.31 | 5.7 ± 1.0 | 3.3 ± 0.5 | 3.2 ± 1.0 | 371.0 ± 276.3 |
| CaHPO₄ | 6 | (0.1-2.0%) | (0.04%) | 6.30 ± 0.05 | 5.9 ± 0.8 | 2.3 ± 0.5 | 3.2 ± 0.4 | 340.0 ± 70.7 |
| Ca₃(PO₄)₂ | 6 | (0.1-2.0%) | (0.04%) | 6.35 ± 0.06 | 5.8 ± 0.4 | 3.0 ± 0.8 | 3.2 ± 1.2 | 515.1 ± 291.6 |
| KH₂PO₄ | 6 | (0.1-2.0%) | (0.04%) | 5.99 ± 0.13 | 5.0 ± 1.3 | 2.2 ± 1.5 | 3.2 ± 0.4 | 396.2 ± 143.8 |
| Ca Lactate | 2 | (0.25%) | (0.04%) | 5.85 ± 0.03 | 5.0 ± 1.4 | — | 3.0 ± 1.4 | 657.0 ± 360.2 |
| Mg(OH)₂ | 8 | (0.1-2.0%) | (0.04%) | 8.73 ± 0.60 | 5.3 ± 1.6 | 2.0 ± 0.7 | 3.0 ± 1.7 | 482.7 ± 264.5 |
| Zn Gluconate | 2 | (0.25%) | (0.04%) | 5.92 ± 0.07 | 5.0 ± 0.0 | 1.0 ± 0.0 | 3.0 ± 1.4 | 366.7 ± 92.4 |
| MgO | 10 | (0.1-2.0%) | (0.04%) | 6.59 ± 0.67 | 5.8 ± 0.6 | 3.5 ± 0.5 | 2.8 ± 0.6 | 458.3 ± 105.4 |
| MgHPO₄ | 6 | (0.1-2.0%) | (0.04%) | 6.41 ± 0.18 | 5.4 ± 0.5 | 1.3 ± 0.5 | 2.7 ± 1.2 | 522.0 ± 150.8 |
| Ca Gluconate | 2 | (0.25%) | (0.04%) | 6.04 ± 0.13 | 6.0 ± 1.4 | — | 2.5 ± 2.1 | 618.0 ± 331.6 |
| CaCl₂ | 14 | (0.1-2.0%) | (0.0-0.04%) | 6.59 ± 0.87 | 4.7 ± 1.0 | 1.4 ± 1.1 | 2.3 ± 0.8 | 348.5 ± 300.3 |
| MgCl₂ | 2 | (0.25%) | (0.04%) | 5.97 ± 0.19 | 5.5 ± 0.7 | — | 2.0 ± 1.4 | 596.0 ± 311.1 |
| Na₃PO₄ | 6 | (0.1-2.0%) | (0.04%) | 7.28 ± 0.85 | 5.8 ± 2.3 | 4.3 ± 1.0 | 1.8 ± 1.0 | 171.1 ± 140.0 |
| K₂HPO₄ | 6 | (0.1-2.0%) | (0.04%) | 6.74 ± 0.36 | 5.7 ± 0.6 | 4.0 ± 1.2 | 1.8 ± 1.3 | 232.0 ± 74.8 |
| Mg Citrate | 6 | (0.1-2.0%) | (0.04%) | 6.10 ± 0.13 | 4.8 ± 0.6 | 1.0 ± 0.0 | 1.7 ± 0.8 | 421.9 ± 310.7 |
| CaO | 5 | (0.1-2.0%) | (0.04%) | 9.69 ± 1.63 | 4.0 ± 2.5 | 1.8 ± 1.3 | 1.6 ± 0.9 | 5075.0 ± 4569.1 |
| Ca Citrate | 6 | (0.1-2.0%) | (0.04%) | 6.01 ± 0.09 | 5.6 ± 0.8 | 1.3 ± 0.5 | 1.5 ± 0.8 | 549.0 ± 567.1 |
| K₂CO₃ | 6 | (0.1-2.0%) | (0.04%) | 8.31 ± 1.17 | 4.8 ± 2.3 | 2.8 ± 2.1 | 1.3 ± 0.8 | 202.0 ± 91.4 |
| Ca(H₂PO₄)₂ | 5 | (0.1-2.0%) | (0.04%) | 5.65 ± 0.40 | 4.8 ± 2.3 | 1.2 ± 0.4 | 1.2 ± 0.4 | 227.9 ± 104.8 |
| KOH | 3 | (0.01-0.05%) | (0.04%) | 7.24 ± 0.23 | 6.3 ± 1.5 | 4.0 ± 0.0 | 1.0 ± 0.0 | 221.0 ± 105.2 |
| Na₂CO₃ | 6 | (0.1-2.0%) | (0.04%) | 8.53 ± 1.08 | 4.0 ± 2.8 | 2.3 ± 2.6 | 1.0 ± 0.0 | 254.3 ± 70.9 |

Table 4 generally shows that as the general concentration of ionic compounds, including monovalent and multivalent cations, increases as used in the process of the present disclosure, functional characteristics of the formulation change substantially. Milk sensory quality decreases as concentration of ionic compounds increases. Foam quality also decreases as the concentration of ionic compounds increases. Feathering also becomes more apparent as concentration of ionic compounds increases beyond a certain point.

A preferred concentration of ionic compounds according to the present disclosure may be, in some embodiments, between 0.05 and 0.15%, when combined with proteases at certain concentrations. In accordance with the present disclosure, and without being bound by theory, the presence of ionic compounds may counter the increase in acidity caused by hydrolysis caused by protease activity. The presence of ionic compounds, particularly ionic compounds that dissociate gradually such as calcium carbonate, may be important to the effectiveness of the process of the present disclosure. As shown in the Tables, many combinations of divalent cations, monovalent cations, and multivalent cations with trypsin are not effective with regard to the present disclosure. While the reason that certain divalent cations, such as calcium carbonate, are effective while other divalent cationic salts, such as magnesium carbonate, are less effective is unknown, the data included in the present disclosure show that this difference is practically and statistically significant with regard to use in plant based beverage products.

TABLE 4
Effects of concentration of ionic compounds on oat milk and creamer

| | | | | Milk | | | |
|---|---|---|---|---|---|---|---|
| Ionic | | Quantity of | | Sensory | Foam | Feathering | Creamer |
| Compounds | n | Proteases | pH of Milk | Quality | Quality | in Coffee | Viscosity (cPs) |
| (0.05-0.15%) | 29 | (0.01-0.3%) | 6.59 ± 0.41 | 6.3 ± 0.9 | 2.9 ± 1.1 | 2.9 ± 1.1 | 326.2 ± 211.8 |
| (0.4-0.55%) | 33 | (0.01-0.2%) | 7.13 ± 0.93 | 5.8 ± 1.1 | 2.5 ± 1.2 | 2.7 ± 1.3 | 590.6 ± 1697.1 |
| (1.0-1.25%) | 33 | (0.01-0.2%) | 7.37 ± 1.14 | 5.3 ± 1.6 | 2.4 ± 1.3 | 2.4 ± 1.1 | 567.2 ± 1382.7 |
| (1.5-2.5%) | 30 | (0.01-0.3%) | 7.54 ± 1.35 | 4.7 ± 1.9 | 1.9 ± 1.3 | 2.2 ± 1.1 | 548.0 ± 597.5 |

Table 5 shows that some proteases are effective in the process of the present disclosure while others less effective. The combination of trypsin and calcium carbonate containing compounds provides good milk sensory quality, good foamability and a high reduction in feathering. Feathering is more pronounced when neutral protease or papain are used in the process of the present disclosure. Further, neutral protease and papain are less effective in maintain or increasing milk sensory quality, when compared to trypsin or alkaline protease.

TABLE 5
Effects of different proteases on oat milk and creamer

| | | Ionic | | | | | |
|---|---|---|---|---|---|---|---|
| Type of | | Compounds | | Milk | | | |
| Proteases | | Added | | Sensory | Foam | Feathering | Creamer |
| (uantity) | n | (Quantity) | pH of Milk | Quality^{€} | Quality^{ϕ} | in Coffee^{¶} | Viscosity (cPs) |
| Trypsin | 2 | CaCO₃, MgO | 6.68 ± 0.64 | 7.5 ± 0.7 | 4.0 ± 0.0 | 4.5 ± 0.7 | 290.0 ± 268.7 |
| (0.04%) | | (0.25%) | | | | | |
| Alkaline | 2 | CaCO₃, MgO | 7.76 ± 0.96 | 7.0 ± 1.4 | 3.5 ± 0.7 | 4.0 ± 1.4 | 332.0 ± 267.8 |
| Protease | | (0.25%) | | | | | |
| (0.04%) | | | | | | | |
| Neutral Protease | 2 | CaCO₃, MgO | 6.76 ± 0.47 | 5.5 ± 0.7 | 4.0 ± 0.0 | 3.5 ± 0.7 | 309.3 ± 207.4 |
| (0.04%) | | (0.25%) | | | | | |
| Papain | 2 | CaCO₃, MgO | 6.86 ± 0.52 | 6.5 ± 0.7 | 3.5 ± 0.7 | 3.0 ± 0.0 | 358.0 ± 14.1 |
| (0.04%) | | (0.25%) | | | | | |

Table 6 shows the effect of trypsin concentration on oat milk and oat creamer, in accordance with the present disclosure. Table 6 shows that, in general, trypsin concentration can be optimized in the context of the present disclosure to produce optimal results. Trypsin concentration may be most effective between 0.04% and 0.08% for some purposes, however, in some embodiments, desired results may result from concentrations outside this range.

TABLE 6
Effect of trypsin concentration with calcium carbonate on oat milk
and oat creamer

| Amount of | | | | Milk | | | |
|---|---|---|---|---|---|---|---|
| Trypsin | | Quantity of | | Sensory | Foam | Feathering | Creamer |
| (%) | n | CaCO₃ | pH of Milk | Quality | Quality | in Coffee | Viscosity (cPs) |
| 0.01 | 6 | (0.1-2.0%) | 7.15 ± 0.31 | 6.3 ± 0.5 | 3.8 ± 0.4 | 2.3 ± 0.5 | 325.4 ± 60.1 |
| 0.02 | 2 | (0.5-1.0%) | 7.28 ± 0.16 | 6.3 ± 0.4 | 3.5 ± 0.7 | 3.0 ± 0.0 | 280.7 ± 21.7 |
| 0.03 | 2 | (0.5-1.0%) | 7.13 ± 0.02 | 5.5 ± 0.7 | 3.0 ± 0.0 | 3.0 ± 0.0 | 254.3 ± 31.6 |
| 0.04 | 27 | (0.05-2.5%) | 7.17 ± 0.30 | 6.7 ± 0.9 | 3.6 ± 0.7 | 4.1 ± 1.1 | 260.5 ± 90.6 |
| 0.05 | 7 | (0.25-2.0%) | 7.14 ± 0.26 | 7.1 ± 0.7 | 2.7 ± 0.5 | 2.4 ± 0.5 | 276.0 ± 72.9 |
| 0.08 | 2 | (0.5-1.0%) | 7.23 ± 0.30 | 7.0 ± 0.7 | 3.5 ± 0.7 | 4.0 ± 0.0 | 243.7 ± 38.2 |
| 0.10 | 6 | (0.1-1.5%) | 7.14 ± 0.29 | 6.3 ± 1.2 | 2.3 ± 0.5 | 3.3 ± 0.5 | 305.2 ± 57.6 |
| 0.20 | 6 | (0.25-2.0%) | 7.14 ± 0.15 | 6.5 ± 0.8 | 1.3 ± 0.5 | 3.8 ± 0.8 | 469.9 ± 161.7 |
| 0.30 | 4 | (0.25-2.0%) | 7.05 ± 0.35 | 6.5 ± 0.0 | 1.0 ± 0.0 | 4.3 ± 1.0 | 532.5 ± 62.8 |

Table 7 shows the effect of CaCO3 concentration on the process of the present disclosure. The data from Table 7 shows the effect of divalent cation concentration on the process of the present disclosure, such that calcium carbonate concentration may optimally reduce feathering at a concentration of approximately 0.3%. Without being bound by theory, calcium carbonate may help to maintain pH in the appropriate range for enzyme activity, whereas calcium hydroxide alone may increase pH too rapidly for effective hydrolysis and feathering reduction through structural changes to hydrolysates.

TABLE 7
Effect of concentration of Calcium Carbonate in Trypsin treated oat milks
and their formulated creamers

| Amount of | | Quantity of | | | Foam | Feathering | |
|---|---|---|---|---|---|---|---|
| CaCO₃ (%) | n | Trypsin | pH of Milk | Milk Quality^{€} | Quality^{ϕ} | in Coffee^{¶} | Viscosity (cPs) |
| 0.1 | 5 | (0.01-0.3%) | 6.65 ± 0.06 | 6.9 ± 0.4 | 2.8 ± 1.3 | 3.6 ± 1.3 | 271.5 ± 131.9 |
| 0.2 | 2 | (0.04%) | 7.04 ± 0.18 | 6.3 ± 0.4 | — | 2.0 ± 0.0 | 352.7 ± 66.9 |
| 0.3 | 13 | (0.04-0.3%) | 7.03 ± 0.11 | 6.9 ± 0.8 | 2.7 ± 1.4 | 4.6 ± 0.9 | 270.6 ± 161.1 |
| 0.5 | 14 | (0.01-0.2%) | 7.09 ± 0.10 | 6.6 ± 0.8 | 2.9 ± 0.9 | 3.6 ± 0.9 | 282.0 ± 119.2 |
| 1.0 | 14 | (0.01-0.2%) | 7.33 ± 0.10 | 6.4 ± 1.2 | 2.8 ± 1.0 | 3.2 ± 1.0 | 335.8 ± 104.9 |
| 1.5 | 5 | (0.04-0.3%) | 7.39 ± 0.16 | 6.5 ± 0.4 | 2.0 ± 1.0 | 3.0 ± 0.7 | 387.5 ± 82.7 |
| 2.0 | 6 | (0.01-0.3%) | 7.43 ± 0.13 | 6.3 ± 0.6 | 2.0 ± 1.4 | 2.8 ± 0.8 | 371.7 ± 59.1 |
| 2.5 | 2 | (0.04%) | 7.54 ± 0.09 | 6.8 ± 0.4 | 4.0 ± 0.0 | 4.0 ± 1.4 | 307.3 ± 51.9 |

Table 8 shows that CaCbTRY1 reduces viscosity of oat milk to a greater degree than trypsin or calcium carbonate alone.

TABLE 8
Difference in oat milk quality with and without Trypsin and CaCO₃

| | | | | | | Foam | |
|---|---|---|---|---|---|---|---|
| Treatment | | Quantity of | Quantity of | | | Quality^{ϕ} | Milked Oat |
| Abbreviation | n | CaCO₃ | Trypsin | pH of Milk | Milk Quality^{€} | (mL^{¥}) | Viscosity (cPs) |
| TRY1 | 8 | 0.00% | (0.02-0.2%) | 6.14 ± 0.13 | 4.9 ± 0.5^{c} | 4.9 ± 0.4^{a} | 50.0 ± 10.0^{a} |
| | | | | | | (247) | |
| CaCbTRY1 | 8 | (0.25%) | (0.02-0.2%) | 7.01 ± 0.13 | 6.8 ± 0.8^{ab} | 2.4 ± 0.5^{c} | 40.1 ± 5.0b^{bc} |
| | | | | | | (134) | |
| CaCb | 8 | (0.25-2.0%) | (0.00%) | 7.37 ± 0.24 | 5.9 ± 0.7^{b} | 3.3 ± 0.7^{b} | 41.3 ± 4.0^{ab} |
| | | | | | | (163) | |
| CaCbTRY1 | 8 | (0.25-2.0%) | (0.04%) | 7.26 ± 0.24 | 7.2 ± 0.7^{a} | 2.1 ± 0.4^{c} | 37.7 ± 5.5^{bc} |
| | | | | | | (137) | |

^{a-c}Represents that different letters in the same column are significantly different according to a two-tailed T-test at p < 0.05.

Table 9 shows the effect of treatment according to the present disclosure on soft serve ice cream.

TABLE 9
Effect of calcium carbonate and trypsin on oat milk quality and
formulated soft serve ice cream

| | Treatments | | | |
|---|---|---|---|---|
| Treatment Abbreviation | TRY1 | CaCb | CaCbTRY1 | None |
| n | 1 | 1 | 1.00 | 1.00 |
| Quantity of CaCO₃ | 0.0% | 1.0% | 1.0% | 0.0% |
| Quantity of Trypsin | 0.1% | 0.0% | 0.1% | 0.0% |
| pH of Milk | 6.25 | 7.62 | 7.47 | 6.65 |
| Milk Quality^{€} | 5.5 | 5.0 | 7.0 | 5.0 |
| Foam Quality^{ϕ}(mL^{¥}) | 5.0 (275) | 3.0 (175) | 2.0 (150) | 4.0 (200) |
| Milk Viscosity (cPs) | 51 | 42 | 35 | 51 |
| Soft Serve Viscosity (cPs) | 463 | 171 | 167 | 343 |
| pH of Soft Serve | 6.06 | 6.98 | 6.93 | 6.16 |
| Soft Serve Quality^{€} | 5.5 | 5.5 | 7.0 | 5.0 |
| Shake Quality^{€} | 5.0 | 5.5 | 7.0 | 5.0 |

Table 10 is a calculation for degree of hydrolysis (DH) with regard the present disclosure. Table 10 shows the relative amounts of protein products above and below 50 kDa measured before and after treatment according to the present disclosure. This was performed with soy milk produced according to the present disclosure.

TABLE 10
Degree of Hydrolysis (DH) in 0.04% Trypsin treated soy protein
concentrate

| | | Relative (%) Quantity | |
|---|---|---|---|
| | | of Peptides of | Degree of |
| | | Molecular Weight | Hydrolysis |
| Treatment | n | below 50 kDa | (%) |
| None, CaCl, CaCb | 6 | 59.8 ± 7.1 | 0 |
| TRY1, CaCbTRY1, | 10 | 68.5 ± 2.1 | 8.7 |
| CaClTRY1, NaClTRY1, | | | |
| KOHTRY1 | | | |

^{€}CaCl₂, CaCO₃, NaCl or no ionic compounds were added to encompass the effect of presence of salts during protein hydrolysis.

In addition to feathering problems, plant based milk is often perceived as having a lower quality taste than dairy milk. Many consumers report that the flavor of plant based milks has undesirable notes, such as bitter, astringent and rancid (McClements, 2021).

For example, plant based coffee creamers, which are made from plant based milk, are far more likely to coagulate, or feather, when combined with coffee. This is perceived negatively by a consumers of product.

Surprisingly, as shown in Table 1, the present disclosure found that the addition of calcium carbonate alone improved the sensory quality of the milk. Calcium carbonate is not generally known as a sensory enhancing compound, and is considered to have a soapy, lemony taste on its own. U.S. Pat. No. 20140044855 to Sher and Bezelgues compared the effects of using calcium carbonate and calcium citrate for whitening in a creamer, and also looked at the effects of calcium citrate on the sensory properties of the creamer. Although no analysis of the effect of calcium carbonate on taste was provided, no change in beverage taste, either positive or negative, was reported by Sher for calcium citrate.

Interestingly, over a concentration range of approximately (0.05-2.5%), and particularly at a concentration of 0.25%, the addition of calcium carbonate to plant based oat milk prepared as shown in Table 2 increased milk sensory quality. In some embodiments, on a hedonic sensory scale of 1.0 to 9.0, the addition of calcium carbonate increased the score from 5.0 to 6.0.

For some applications, such as barista use in foamed coffee beverages, foaming is desirable. For other applications, however, foaming is undesirable. For example, foaming during processing may cause difficulties for process engineers and technicians. Further, for conventional creamer use, such as table top restaurant creamers, foaming may not be desirable.

Creamer viscosity is generally only commercially acceptable at below 500 cPs (at a refrigerated temperature). Therefore, some formulations disclosed in the tables 1-10 herein meet this commercial acceptability standard, while some do not. Some of the formulations of the present disclosure, including calcium carbonate alone in a creamer formulation, the combination of calcium carbonate and protease, calcium hydroxide and calcium chloride combined with the creamer formulation, calcium carbonate and magnesium oxide as well as some other combinations of divalent cationic salts and proteases meet the commercially acceptable viscosity standard of 500 cPs.

In one embodiment, the process according to the present disclosure further includes grinding a mix that includes the raw material, enzyme, and macro-mineral salt using size reduction machinery to make a paste, slurry, or solution to preferably reduce the particle size to smaller than 1 mm in diameter, with the grinding process preferably occurring at below native protein denaturation temperature. In some embodiments, the fiber and hull may be removed from the raw material.

In one embodiment, a slurry containing milled plant material (raw material, proteases, macro-mineral salts, such as calcium carbonate, along with, optionally, amylases, lipases, and other additives) may be heated using a heat exchanger, a kettle with mixing or any kind of heating equipment to achieve heating at a rate of approximately 0.1-50° C. per minute to a temperature beyond the denaturation temperature of the enzymes (typically 100° C. or 220° F.). Here, the macro-mineral salts may dissociate into cations (i.e. Ca++) and the pH of the media increases to slight alkali (˜pH 7.5) as a result of salt dissociation in heated aqueous media; next, enzymes may hydrolyze substrate to native proteins; and next, macro-mineral cations may bind or interact with hydrolyzed and non-hydrolyzed constituents (mainly protein) of raw materials to theoretically allow protein molecules form casein micelle like structure.

Without being bound by theory, in the present invention, macro-mineral cations (i.e. Ca++) may bind protein hydrolysates on acidic (aspartic and glutamic acids), and polar (serine and threonine) amino acids residues and cysteine molecules, thereby creating bonds among hydrolysates and protein networks, thus stabilizing the entire protein system in a manner similar to a casein micelle. The mix may then be cooled down, allowing the protein to refold and stabilize for further application in food or feed formulations.

### Example 1

Oat and brown rice milk were treated according to the present disclosure to create modified plant protein using protease and calcium carbonate.

### Ingredients:

a. 100 g of oat or brown rice.
b. 0.04 g microbial trypsin
c. 0.03 g Bacterial amylase (alpha-amylase)
d. 0.03 g Calcium Chloride (amylase cofactor)

### e. 0.5 g Calcium Carbonate

f. 500 mL ice water (38° F.)

### Procedure:

a. Oat grain (100 g) was washed with ice cold water (38° F.) three times; the wash water was drained using a strainer.
b. Wet grain (˜115 gram with water) was added to a Vita-Mix TurboBlend 4500 blender.
c. 285 mL of ice-cold water (38° F.), 40 miligrams (mg) of Trypsin, 200 mg of Calcium Carbonate and 30 mg of Calcium Chloride were added to the blender cup. Then, the mix was blended at speed 10 setting for 2 minutes.
d. Then, 30 microliters of Bacterial Amylase (Validase, DSM) was added to the blender cup and mixed for another 15 seconds.
e. The slurry was filtered through 120 mesh screen. Then, 100 mL of cold water added to the remaining solid on the screen and blended for 30 s in the blender, and filtered through 120 mesh (washing). Washing was repeated once, for a total of two washings.
f. The fiber portion was discarded, and only the milk portion was processed further.
g. The pH and amount of total solid of the milk was measured and recorded.
h. The milk was slowly warmed up (10° F./minute) to 170° F. in a water bath maintained at around 200° F.
i. Milk was then heated to boil (˜220° F.) in a microwave for approximately 70 s to deactivate the enzymes.
j. To the boiled milk 300 mg of additional calcium carbonate were added to the milk, followed by cooling to approximately 140° F.
k. The milk was homogenized at 2000 PSI using GEA Niro Sovavi homogenizer, and the homogenized milk was placed in a refrigerator for cooling to 38° F.

### Product Examples:

a. Oat, chickpea, and/or rice protein for soft serve ice cream.
b. Chickpea protein for a dairy milk replacement beverage.
c. Oat protein creamer.
e. Rice protein creamer.

### Example 2

Ice cream soft serve manufacturing procedure:
1. Place 50% of culinary water in the formula of soft serve ice cream in hot (115° F.) into a Breddo® mixer.
2. Rotate the mixer blade in the mixer at high speed, keep the mixing blade on, and place the full amount of sunflower lecithin in the formula to the mixer, and mix the blend for 5 minutes.
3. Add the entire amount of heated and melted (115° F.) coconut oil into the mixer, and mix for 5 minutes.
4. Add the entire amount of ambient temperature canola oil into the mixer, and mix for 5 minutes.
5. Add the entire amount of oat or rice concentrate (produced according to the process of the present disclosure) and milked chickpea concentrate, liquid sugar and salt, and mix for additional 10 minutes (in one embodiment, chickpea concentrate is produced according to the process of the present disclosure with the only difference in the protocol being an initial hydrolysis by neutral protease (0.02%) (due to the higher concentration of protein when compared to the oat or rice material), wherein the neutral protease is allowed to act for approximately 2 minutes at optimal activity temperature, followed by neutral protease deactivation by heating up in accordance with steps i and j above; for the chickpea protocol, the trypsin reaction for all other steps, proceed according to steps i and j after the neutral protease reaction).
6. Cool down the mix to 45° F., and transfer the mix into a storage container maintained at 45° F.
7. Place the rest of culinary water (50%) in the formula in ambient temperature to the mixer, and agitate at a high speed setting on the mixer.
8. Add any additional ingredients including flavoring, mix for 5 minutes, and transfer the blend into the storage container and mix with the entire blend in the silo with a low agitation (50% of full speed) in the storage container for a minimum of 2 hours until the base is further processed.
9. Process the soft serve base through a UHT process, and aseptically package into retail packages for distribution and sales.
For chocolate formula, a small portion of water (10%) in the formula is used to make a cocoa slurry (heated to 195° F., kept at 195° F. for 1 hour, cooled to 115° F.) and the cocoa slurry is added to the Step 2 of the manufacturing procedure prior to add the sunflower lecithin.
Soft serve (vanilla) formulation:

### Description:

Culinary water: 10-90% w/w

### Sunflower Lecithin: 0.001-10% w/w

### Coconut oil: 0.5-50% w/w

### Canola oil: 0.5-50% w/w

### Oat Concentrate: 0.1-50% w/w

### Chickpea Concentrate: 0.1-50% w/w

### Sugar, Liquid: 1-80% w/w

### Salt: 0.01-5% w/w

### Natural Flavors: 0.001-20% w/w

cocoa powder (only for chocolate formula): 0.1-50% w/w

Plant based milk is the main ingredient in the plant based creamer of the present disclosure. Milk quality was evaluated using a 9 point quality scale to measure sensory properties; 1 indicating low quality milk having numerous off notes and 9 indicating high quality milk having no off notes, high flavor intensity, a desired level of sweetness; good mouthfeel and good color.

Foam quality deteriorates with certain proteases and ionic compounds, particularly calcium carbonate and trypsin. Foam reduction has value for certain applications of the present disclosure. Foam reduction is unexpected and synergistic according to the process of the present disclosure, as is observed with the combination of calcium carbonate and trypsin, a preferred embodiment.

The foam quality and volume of milked oats revealed interesting aspects of the protease hydrolysis in the presence and absence of multivalent cations, including calcium, as shown in Tables 7 and 8. The results indicated that plant milks treated with trypsin resulted in excellent foam quality and volume. In contrast, the foam quality of trypsin treated in the presence of calcium carbonate (CaCbTRY1) showed the least amount of foaming. Interestingly, the foam quality, or foam suppression, by calcium carbonate was also observed in the non-protease treated milk, but the degree of suppression was much lower than that in protease treated milks. The foam quality and volume of CaCbTRY1 would be expected to be similar or better than untreated milk or and CaCb, however, it was not.

In addition, it was observed that the ice cream soft serve base from TRY1 only was too viscous, and thus had flowability problems in a gravity fed soft serve ice cream machine. The viscosity of soft serve ice cream base with untreated milk, wherein untreated milk refers to the absence of treatment with protease and divalent cationic salts according to the present disclosure, is of borderline acceptability, however, this borderline acceptability will likely become unacceptable over the course of its shelf life due to the tendency of soft serve ice cream base becoming thicker over time. Normally, the viscosity of soft serve ice cream at the time of manufacture is least viscous, then grows thicker and normalizes. The Viscosity of the CaCb treated soft serve ice cream base is acceptable, but could be more robust if the viscosity were lower. The quality and functionality of soft serve ice cream using of CaCbTRY1 was the highest. The viscosity of CaCbTRY1 milk and soft serve ice cream base would be expected to be higher than CaCb and untreated milk, considering that the viscosity of TRY1 only has the highest viscosity of all tested samples. Surprisingly, the viscosity of CaCbTRY1 was the lowest, which is a result of unexpected, synergistic effects.

In the milk and protein foam matrix, proteins may act as surfactants and interact at an interface to create a foam, visco-elastic film which stabilizes gas bubbles. It is well known that temperature, pH, stabilizers, oils, free fatty acids, surfactants and degree of protein hydrolysis affect foamability and stability of foods and proteins, however, the effects of these minerals on foamability are not fully understood.

The present disclosure, in some embodiments, shows suppression on foam quality in CaCbTRY1, which may, without being bound by theory, result from formation of strong bonds between hydrolyzed polypeptides and multivalent cations (i.e. Ca ions), so the bonds prevent polypeptides from unfolding, rearranging and forming visco-elastic films during the foaming process. It is believed the bonding (Peptide-Ca-Peptide or Protein-Ca-Protein) also exists in the “CaCb” treated samples, so it suppressed its foam quality/volume and viscosity in comparison to “None” sample, but the phenomenon was more obvious and pronounced in the CaCbTRY1 vs TRY1. The addition of Ca++ salts into the plant based milking process not only stabilized the protein hydrolysate in hot acidic coffees, but also lowered the viscosity of the creamers and soft serve ice cream bases resulting in superior quality products. Addition of Ca salts into the plant based milking process with endo-proteases resulted in synergistic, unexpected quality improvements in the final product. Also, the milk and formulated products made of CaCbTRY1 showed some masking properties. Undesirable throat grasping/tinkling/irritation/scratching in most of protease only or no protease treated milks and formulated products made of the milks were reduced or eliminated. Also, tongue coating drying astringency in some of the milks and products was strong and persistent, but in the CaCbTRY1 and products comprising it this effect was relatively weak and brief.

### Viscosity Measurement:

A. Homogenized milks, formulated homogenized creamers, or formulated homogenized soft serve bases stored in a refrigerator maintained at 1.1° C. for a minimum of 15 hours were transferred into beakers and placed in a 1.7° C. ice-water bath, and left in the bath for 10 minutes to get samples and the ice-bath temperature equilibrated. The ice bath temperature was monitored and maintained a constant temperature by adding water or ice.
B. A sample beaker was removed one at a time from the sample ice-ice bath, placed into another ice-water bath maintained at 1.7° C. under the viscometer. Then, the viscosity of the sample mix was measured with Brookfield RVT Series Viscometer (Brookfield Engineering Laboratories Inc., Middleboro, Mass.) equipped with #3, 4 or 5 round disk probe while the sample tube was in the ice-water bath. The viscometer speed was either 50 or 100 rpm, and the viscosity was converted into centipoise (cPs) from a table provided by the viscometer manufacturer. Three readings were collected and averaged for a viscosity.

The viscosity was measured at 1.7° C. in an ice water bath to minimize the variation between samples and to minimize viscosity variations particularly rate variation during warming up the refrigerated samples to a higher temperature (i.e. room temperature, 21° C.).

### Foam Quality:

A. pH measured final milks were diluted to 10% solid milk by adding distilled water and blended.
B. One hundred grams (100 g) of each milk was placed in a Nespresso Milk Frother (Nespresso USA Inc., New York, N.Y.), and foamed.
C. Warm foamed samples were placed in 400 mL graduated beakers, and the volume and the quality of foam was observed and recorded.
D. From the volume of the foam/liquid and quality of the foam, the foam quality was converted and rated between 1 and 5.
(1) Poor quality foam: Volume of milk/foam mix after foaming being 100-120 mL and the size of bubbles are big and collapse quickly.
(2) Below average: Volume of milk/foam mix after foaming being 120-150 mL and the size of bubbles are big and collapse quickly.
(3) Average: Volume of milk/foam mix after foaming being 125-175 mL with a mixture of big micro bubbles and collapse moderately.
(4) Above Average: Volume of milk/foam mix after foaming being 150-200 mL with mostly micro foams and collapse slow.
(5) Excellent: Volume of milk/foam mix after foaming being >200 mL with mostly micro foams and collapse slow.

### Creamer Stability:

A. Eleven grams (11 g) of homogenized and cooled (1.1° C.) creamer samples were placed in a 3 oz Solo cups using disposable transfer pipettes.
B. The portion creamers were placed into 89 mL hot (˜77° C.) acidic (<pH 5.0) brewed coffee, which is equivalent to 1 oz creamers into 8 oz coffee while the coffee being stirred with transfer pipette used to weigh out the creamers. The color and other creamer quality were observed, measures, and converted to creamer stability between 1 and 5.
(1) Very unstable: Feathered instantly (<0.25 minutes)
(2) Unstable: Feathered in less than 3 minutes with large coagulations
(3) Average: Feathered in 3-5 minutes after creamer and coffee mixed and undisturbed afterward.
(4) Pseudo Stable: Feathered between 5-10 minutes and the coagulation is very fine in size.
(5) Stable: Stable for over 10 minutes and beyond without any feathering.
Note 2: Seventy grams (70 g) of ground Lavazza Perfectto dark roast coffee (Lavazza Premium Coffees Co., New York, N.Y.) was brewed with 2840 mL (12 cups) of tap water in a Mr. Coffee machine (Model BVMC-DW12-WF, Sunbeam Product Inc., Boca Raton, Fla.), and resulted in 2600 mL (11 cups) of coffee. The brewed coffee was left for a minimum of 5 hours in the coffee maker with the heat on until it was used for creamer evaluation. The coffee pH ranged from 4.63 to 4.95 and the temperature was approximately 77° C.
Organoleptic Evaluation of milks and products:
A. Approximately 30 mL of milks and products with three digit random number assigned was placed in 3 oz Solo cups.
B. Expert panel member(s) evaluated and rated the overall quality of milks and product using 9 point quality scale.
(1) Lowest quality-Highly unacceptable with lots of off flavors and taste aspects such as smells, bitterness, sourness, salty, astringent, throat scratching, darker or different in color, slimy, viscous in texture, etc. In addition, it includes samples with low to no sweetness, lack of intended flavor (i.e. oat flavor in oat milk).
(5) Medium quality: Neither acceptable nor unacceptable
(9) Highest quality: Highly acceptable without off notes, high intensity of intended flavor, right level of sweetness, mouthfeel, and good color.
C. Between samples panel washed palate with distilled water, unsalted saltine crackers, and waited for minimum of 3 minutes until the palate is clean without any residual off notes from the previous sample evaluation.
A. Between samples panel washed palate with distilled water, unsalted saltine crackers, and waited for minimum of 3 minutes until the palate is clean without any residual off notes from the previous sample evaluation.

### Degree of Protein Hydrolysis (DH)

A. Two Hundred grams (200 g) of Soy, which was purchased in a local East Asian store in Buffalo, N.Y. was washed with ice cold water three times and drained.
B. The washed soy and 800 mL of ice cold water was placed into a Vita-Mix TurboBlend 4500 blender, and blended at speed 10/10 setting for 1 minute.
C. The slurry was filtered through a #120 mesh screen. Then, 400 mL of cold water added to the solid and blended for 30 seconds in the blend, and filtered through #120 mesh screens (washing). Repeated the washing one more time.
D. The fiber portion was discarded, and pH and amount of total solid of the milk was measured, and recorded.
E. The milk was centrifuged at 3000 rpm for 10 minutes to separate the insoluble proteins.
F. The cake was recovered from the centrifuge tubes, and weighed. Then, the cake was diluted with 5× amount of water, blended with a hand held mixer for 2 minutes, and centrifuged again at 3000 rpm for 10 minutes. The cake was then diluted again with 5× water based on the cake weight.
G. Then, the base was divide into to 8 equal portions, and chemicals and trypsin were added for each treatment.
1. Intact Untreated: No enzyme or chemical was added
2. 0.5% (125 mg/25 g Soy) of CaCl2
3. 0.5% (125 mg/25 g Soy) CaCO3
4. 0.04% (10 mg/25 g Soy) Trypsin
5. 0.5% (125 mg/25 g Soy) CaCl2 and 0.04% (10 mg/25 g Soy) of Trypsin
6. 0.24% (60 mg/25 g Soy) NaCl and 0.04% (10 mg/25 g Soy) of Trypsin
7. 25 mg KOH (pH to 7.8) and 0.04% (10 mg/25 g Soy) of Trypsin
8. 0.5% (125 mg/25 g Soy) CaCO3 and 0.04% (10 mg/25 g Soy) of Trypsin.
H. The mix was slowly warmed up to 55° C. in a water bath maintained at around 55° C., and left at 55° C. for 60 minutes to get the protein hydrolyzed.
I. The milk was then heated to 98° C. in a microwave for approximately 70 seconds. The samples were cooled to 4.5° C. for analysis.
J. Total solid and protein content were measured using an Ohaus MB90 Moisture analyzer (Parsippany, N.J.), and by a Dumas method using a NDA 701 Dumas Nitrogen Analyzer (Velp Scientific, Inc., Bohemia, N.Y.) using a conversion factor 6.25.”
K. The samples were diluted to a protein concentration of 4 mg/mL, then dissolved in an equal volume of sodium dodecyl sulfate-polyacrylamide gel electrophoresis (SDS-PAGE) sample buffer, with or without 2-mercaptoethanol (β), and heated in a boiling water for 3 minutes.
L. After cooling of the samples to room temperature, the solutions were centrifuged at 2000×g for 5 minutes to remove non-protein particles.
M. SDS-PAGE gels (separating gel: 12% acrylamide; stacking gel: 5% acrylamide) were prepared based on an established procedures, and the electrophoresis was performed also using a developed procedure in the lab performed the SDS-PAGE analysis.
N. Molecular weight standards were purchased from Sigma-Aldrich Co. All chemical reagents and organic solvents were purchased form Sigma-Aldrich. Quantification of individual protein bands (pixel and %) was done from the SDS-PAGE images using a digitizing analysis software.
O. The Degree of Hydrolysis were determined from the relative quantity changes (% increase) of the peptide quantity having molecular weight less than 50 kDa in ONLY 2-mercaptoethnol added gels.
P. The procedures (A to O) were performed twice to get the Degree of Hydrolysis Abbreviations:

### 1. Trypsin-Microbial: TRY1

### 2. Neutral Protease-L-Bacterial: NEUT

### 3. Papain-Papaya: PAPN

### 4. Alkaline-Protease-Bacterial: ALKP

### 5. Calcium Carbonate: CaCb

### 6. Calcium Hydroxide: CaHy

### 7. Calcium Oxide: CaO

### 8. Calcium Chloride: CaCl

### 9. Calcium Citrate: CaCt

### 10. Calcium Gluconate: CaGl

### 11. Calcium Lactate: CaLt

### 12. Calcium Phosphate Monobasic: MCP

### 13. Calcium Phosphate Dibasic: DCP

### 14. Calcium Phosphate Tribasic: TCP

### 15. Magnesium Carbonate: MgCb

### 16. Magnesium Hydroxide: MgHy

### 17. Magnesium Oxide: MgO

### 18. Magnesium Chloride: MgCl

### 19. Magnesium Citrate: MgCt

### 20. Magnesium Gluconate: MgGl

### 21. Magnesium Phosphate Dibasic: DM

### 22. Sodium Carbonate: NaCb

### 23. Sodium Chloride: NaCl

### 24. Sodium Gluconate: NaGl

### 25. Sodium Phosphate Tribasic: TSP

### 26. Potassium Carbonate: KCb

### 27. Potassium Hydroxide: KOH

### 28. Potassium Phosphate Monobasic: MPP

### 29. Potassium Phosphate Dibasic: DPP

### 30. Aluminum Hydroxide: AlHy

### 31. Zinc Gluconate: ZnGl

### Materials:

A. Proteases used in the experiment:

1. Trypsin-Microbial (TRY1); (Biocat)

2. Neutral Protease-L: Bacterial (NEUT) (Biocat)

3. Papain-Papaya (PAPN) (Biocat)

4. Alkaline-Protease: Bacterial (ALKP) (Biocat)

B. The Quantity of proteases used in the experiment:

0.01-0.3% to the raw material

C. Amylases & their quantity used in the experiment:

1. Bacterial amylase: 0.015-0.06% top the as-is raw material (DSM)

2. Fungal amylase: 0.04% to the as-is raw material (BioCat)

D. Quantity of chemical compounds added into the experiment:

0.00-2.0% to the as-is raw material

E. Chemicals used in the experiment:

1. Calcium compounds: Name (formula)—Abbreviations
a. Calcium Carbonate (CaCO3):CaCb—
b. Calcium Hydroxide (Ca(OH)2): CaHy: (Fisher Chemical, Fair Lawn, N.J.)
c. Calcium Oxide (CaO): CaO (Fisher Chemical, Fair Lawn, N.J.)
d. Calcium Chloride (CaCl2): CaCl
e. Calcium Citrate (Ca3(C6H5O7)2-4H2O): CaCt (Spectrum Chemical Mfg Co., Gardena, Calif.)
f. Calcium Gluconate (C12H22CaO14): CaGl (Acros Organics, Fair Lawn, N.J.)
g. Calcium Lactate (C6H10CaO6):CaLt (Junbunzlauer, Newton, Mass.)
h. Calcium Phosphate Monobasic (CaH4P2O8):MCP (Thermo Fisher Scientific, Ward Hill, Mass.)
i. Calcium Phosphate Dibasic(CaHPO4): DCP—(Loudwolf Industrial & Sci., Dublin, Calif.)
j. Calcium Phosphate Tribasic (Ca3(PO4)2): TCP—(Loudwolf Industrial & Sci., Dublin, Calif.)

2. Magnesium compounds: Name (formula)—Abbreviations
a. Magnesium Carbonate (MgCO3): MgCb (Spectrum Chemical Mfg Co., Gardena, Calif.)
b. Magnesium Hydroxide (Mg(OH)2):MgHy (Fisher Chemical, Fair Lawn, N.J.)
c. Magnesium Oxide (MgO): MgO (Fisher Chemical, Fair Lawn, N.J.)
d. Magnesium Chloride (MgCl2): MgCl (Spectrum Chemical Mfg Co., Gardena, Calif.)
e. Magnesium Citrate (C12H28Mg3O23): MgCt (Stauber, Fullerton, Calif.)
f. Magnesium Gluconate (C12H22MgO14): MgGl (Stauber, Fullerton, Calif.)
i. Magnesium Phosphate Dibasic(HMgPO4): DMP (Fisher Chemical, Fair Lawn, N.J.)

3. Sodium compounds: Name (formula)—Abbreviations
a. Sodium Carbonate (Na2CO3): NaCb—(Loudwolf Industrial & Sci., Dublin, Calif.)
b. Sodium Chloride (NaCl): NaCl (Fisher Chemical, Fair Lawn, N.J.)
c. Sodium Gluconate (C6H1NaO7): NaGl (Acros Organics, Fair Lawn, N.J.)
d. Sodium Phosphate Tribasic (Na3PO4): TSP—Eisen-Golden Laboratories (Dublin, Calif.)

4. Potassium compounds: Name (formula)—Abbreviations
a. Potassium Carbonate (K2CO3):KCb (Spectrum Chemical Mfg Co., Gardena, Calif.)
b. Potassium Hydroxide (KOH):KOH (Spectrum Chemical Mfg Co., Gardena, Calif.)
c. Potassium Phosphate Monobasic (KH2PO4): MPP (Fisher Chemical, Fair Lawn, N.J.)
d. Potassium Phosphate Dibasic(K2HPO4): DPP—Eisen-Golden Laboratories (Dublin, Calif.)

5. Aluminum compound: Name (formula)—Abbreviations
a. Aluminum Hydroxide (Al(OH)3): AlHy (Thermo Fisher Scientific, Ward Hill, Mass.)

6. Zinc compound: Name (formula)—Abbreviations
a. Zinc Gluconate (C12H22O14Zn):ZnGl (Thermo Fisher Scientific, Ward Hill, Mass.)

Trypsin-Microbial, Bacterial Neutral Protease, Papain-Papaya, Alkaline-Protease-Bacterial and Fungal Amylase were obtained from Bio-Cat (Troy, Va.). Bacterial amylase was purchased from DSM (Parsippany, N.J.). Calcium Carbonate (CaCO3) was purchased from Specialty Minerals Inc. (Adams, Mass.). Calcium Hydroxide (Ca(OH)2), Calcium Oxide (CaO), Magnesium Hydroxide (Mg(OH)2), Magnesium Oxide (MgO), Magnesium Phosphate Dibasic(HMgPO4), Sodium Chloride (NaCl) and Potassium Phosphate Monobasic (KH2PO4) were purchased from Fisher Chemical (Fair Lawn, N.J.). Calcium Chloride (CaCl2) was purchased from Avantor Performance Material Inc. (Center Valley, Pa.). Calcium Citrate (Ca3(C6H5O7)2-4H2O) and Calcium Lactate (C6H10CaO6) were obtained from Junbunzlauer (Newton, Mass.). Calcium Gluconate (C12H22CaO14) and Sodium Gluconate (C6H1NaO7) were purchased from Acros Organics (Fair Lawn, N.J.). Calcium Phosphate Dibasic (CaHPO4), Calcium Phosphate Tribasic (Ca3(PO4)2) and Sodium Carbonate (Na2CO3) were supplied by Loudwolf Industrial & Science (Dublin, Calif.). Magnesium Carbonate (MgCO3), Magnesium Chloride (MgCl2) and Potassium Carbonate (K2CO3) were supplied by Spectrum Chemical Mfg. Co. (Gardena, Calif.). Magnesium Citrate (C12H28Mg3O23) and Magnesium Gluconate (C12H22MgO14) were supplied by Stauber (Fullerton, Calif.). Sodium Phosphate Tribasic (Na3PO4) and Potassium Phosphate Dibasic (K2HPO4) were obtained from Eisen-Golden Laboratories (Dublin, Calif.). Potassium Hydroxide (KOH) was obtained from Mallinckrodt Pharmaceuticals (Hampton, N.J.). Aluminum Hydroxide (Al(OH)3), Calcium Phosphate Monobasic (CaH4P2O8) and Zinc Gluconate (C12H22O14Zn) was purchased from Thermo Fisher Scientific (Ward Hill, Mass.).

Having described embodiments of the present disclosure, it is to be understood that the invention may otherwise be embodied within the scope of the appended claims. Although the disclosure has been described with reference to certain preferred embodiments, it will be appreciated by those skilled in the art that modifications and variations may be made without departing from the spirit and scope of the disclosure. It should be understood that applicant does not intend to be limited to the particular details described above and illustrated in the accompanying drawings. It is to be understood that while the invention has been described in conjunction with the detailed description thereof, the description provided herein is intended to illustrate and not limit the scope of the invention, which is defined by the scope of the appended claims. Other aspects, advantages, and modifications are within the scope of the following claims.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A composition comprising:
a synergistic combination of a plant material, an endoprotease and a divalent cationic salt;
wherein the plant material includes at least one plant protein;
wherein the composition comprises a hydrolyzed plant based liquid; and
wherein the hydrolyzed plant based liquid has synergistically improved functionality in at least one of feathering, foaming or organoleptic properties.

17. The composition of claim 16, wherein the hydrolyzed plant based liquid comprises a base for producing a plant based product.

18. The composition of claim 17, wherein the plant based product comprises at least one of a plant based creamer, a plant based ice cream or any other dairy counterpart product.

19. The composition of claim 18, wherein when the hydrolyzed plant based liquid is used in a plant based creamer and a synergistic effect on reduction of feathering in a highly acidic beverage is observed;
wherein a viscosity of the composition is below 500 cPs; and
wherein a starting pH of the highly acidic beverage is below 5.0 prior to adding the plant based creamer.

20. The composition of claim 19, wherein the divalent cationic salt is calcium carbonate.

21. The composition of claim 19, wherein the endoprotease is selected from a group consisting of trypsin and alkaline protease.

22. The composition of claim 16, wherein the plant material is selected from a group consisting of grains, nuts and seeds.

23. The composition of claim 16, wherein a range of the endoprotease is between 0.01% to 0.3% w/w of a raw plant material, and a range of an amount of cation in the divalent cationic salt is between 0.02% to 1.2% w/w of the raw plant material; and
wherein a degree of hydrolysis is low, approximately in a range of between about 3% to 15%.

24. A process, comprising:
grinding a plant based material;
forming a plant based liquid with the plant based material, wherein the plant based liquid is aqueous;
adding a divalent cationic salt to the plant based liquid;
adding an endoprotease to the plant based liquid contemporaneously or after addition of the divalent cationic salt; and
forming a hydrolyzed plant based liquid.

25. The process of claim 24, further comprising wet milling the plant based material.

26. The process claim 24, further comprising selected the endoprotease from a group consisting of trypsin and alkaline protease.

27. The process of claim 24, wherein the divalent cationic salt is calcium carbonate.

28. The process of claim 24, wherein the divalent cationic salt is at least one of calcium carbonate, calcium carbonate combined with a different cationic divalent salt or calcium hydroxide combined with calcium chloride.

29. The process of claim 24, wherein a range of the endoprotease is between 0.01% to 0.3% w/w of a raw plant material, and a range of an amount of cation in the divalent cationic salt is between 0.02% to 1.2% w/w of the raw plant material, and a degree of hydrolysis is low, approximately in a range of between about 3% to 15%.

30. The process of claim 24, wherein when the hydrolyzed plant based liquid is used in a plant based creamer and a synergistic effect on reduction of feathering in a highly acidic beverage is observed;
wherein a viscosity of the plant based creamer is below 500 cPs; and
wherein a starting pH of the highly acidic beverage is below 5.0.

31. The process of claim 24, wherein the plant based material is oat grain.

32. A process, comprising:
grinding a plant based material;
forming a plant based liquid with the plant based material, wherein the plant based liquid is aqueous;
adding a divalent cationic salt to the plant based liquid;
adding an endoprotease to the plant based liquid contemporaneously or after addition of the divalent cationic salt;
forming a hydrolyzed plant based liquid;
sifting the hydrolyzed plant based liquid; and
heating the hydrolyzed plant based liquid to deactivate the endoprotease.

33. The process of claim 32, wherein a range of the endoprotease is between 0.01% to 0.3% w/w of a raw plant material, and a range of an amount of cation in the divalent cationic salt is between 0.02% to 1.2% w/w of the raw plant material; and
wherein a degree of hydrolysis is low, approximately in a range of between about 3% to 15%.

34. The process of claim 32, wherein the hydrolyzed plant based liquid has a synergistically lower viscosity to solids content ratio than without a divalent cationic salt and endoprotease combination.

35. The process of claim 32, wherein when the hydrolyzed plant based liquid is used in a plant based creamer, a synergistic effect on reduction of feathering in a highly acidic beverage is observed;
wherein a viscosity of the plant based creamer is below 500 cPs; and
wherein a starting pH of the highly acidic beverage is below 5.0.
